(19) United States
(12) Patent Application Publication
(10) Pub. No.: US 2007/0282630 A1
(43) Pub. Date: Dec. 6, 2007

(54) Integrated owner-member medical retreat method & system

(76) Inventor: Claus Peter Roehr, Palm Coast, FL (US)

Correspondence Address: William M. Hobby, III, 157 E. New England Avenue, # 375, Winter Park, FL, 32789, US

(21) Appl. No.: 11/800,745

(22) Filed: May 8, 2007

Related U.S. Application Data
(60) Provisional application No. 60/810,698, filed on Jun. 2, 2006.

(51) Int. Cl. G06Q 10/00; A61B 5/00

(52) U.S. Cl. 705/2; 600/300

(57) ABSTRACT

An integrated medical clinic and retreat owner-member treatment process includes the steps of acquiring an equity ownership in a medical clinic and membership in a clinic associated medical retreat program to become an owner-member. The owner-member starts the retreat program with the process of entering the medical clinic for a doctor's examination and for diagnostic testing. The diagnostic testing and the doctor's examination information are processed in a central computer for designing an owner-member wellness program for entering the medical retreat program using diagnostic test results and a doctor's examination and including designing custom nutrition activities and therapy plans for the owner-member during his tenure in the medical retreat.

This application claims the benefit of U.S. Provisional Application No. 60/810,698, filed Jun. 2, 2006.

### FIELD OF THE INVENTION

The present invention relates generally to a method and system for providing a comprehensive integrated medical clinic and retreat for improving the general health and well being of owner-members of the clinic and retreat. A medical clinic and retreat is set up in the nature of a club having equity memberships and an integrated medical rejuvenation center which features a diagnostic, scientific and medical clinic having custom alternative medical choices, a gymnasium and fitness center, and a spa and beauty treatment facility. Members can complete a medical and diagnostic checkup with a comprehensive physical exam and testing which is integrated with personal preferences for nutrition, activities and therapy in order to structure a member's personal lifestyle. The system brings together all of the facilities and health professionals to motivate and educate and advise each member for a longer and healthy lifestyle.

### BACKGROUND OF THE INVENTION

Presently most health care providers are generally only able to offer isolated and specialized care targeting an individual's specific maladies rather than offering a comprehensive approach to the individual's health and general well being. This isolated approach does not take into account the individual's lifestyle including his normal diet and physical activities and integrate this with personal medical history and diagnostic testing and a physical exam to institute a program to improve the individual's health and longevity. A retreat for advising and altering an individual's lifestyle and then providing diagnostic tests to show the results of the changed lifestyle is not provided by health care providers as part of an overall health program. For instance, a nutritional analysis requires the collection of comprehensive dietary intake information for effective use by a dietitian analyst for use in developing a diet of specific foods, quantities and the like but which is tailored to the individual's preferences. Other acts of a retreat program requires the analysis of an individual's normal activities and sports in order to tailor a program for that individual which provides the exercise and activities for a healthy lifestyle. Dynamic management of the health care, nutrition and well being of individuals requires the use of an individual personal medical history along with diagnostic tests of the individual brought together with his normal nutrition and activities and providing a regimen which is then put into effect for a period of time and providing the exact diagnostic tests and then making a determination of the benefits derived from the program.

### SUMMARY OF THE INVENTION

An integrated medical clinic and retreat owner-member treatment process includes the steps of acquiring an equity ownership in a medical clinic and membership in a clinic associated medical retreat program to become an owner-member. The owner-member starts the retreat program with the process of entering the medical clinic for a doctor's examination and for diagnostic testing. The diagnostic testing and the doctor's examination information are processed in a central computer for designing an owner-member wellness program for entering the medical retreat program using diagnostic test results and a doctor's examination and including designing custom nutrition activities and therapy plans for the owner-member during his tenure in the medical retreat. The owner-member then enters the medical retreat program for a predetermined period of time, such as three to six weeks, to follow a design wellness program custom designed for the owner-member. Exit diagnostic testing is used for evaluating the owner-member's change during the medical retreat so that a person can improve his health through an owner-membership program in a medical clinic and associated medical retreat program. The process also include the step of referring the owner-member to a specialist responsive to its diagnostic testing results prior to entering the medical retreat program upon discovery of more serious medical problems. The designing of an owner-member wellness program includes physical activity formulated in accordance with the owner-member's interest to provide the necessary physical activity. The owner-member's nutrition regimen is formulated in accordance with the owner-member's taste. The process also includes designing an owner-member wellness program including a motivational program and designing an alternative therapy program.

### BRIEF DESCRIPTION OF THE DRAWINGS

Other objects, features, and advantages of the present invention will be apparent from the written description and the drawings in which:

FIG. 1 is a block diagram of a medical retreat integrated with a gymnasium and activity center which is connected to nearby entertainment and residences and to a commercial area having restaurant, entertainment, and lodging;

FIG. 2 is a schematic diagram of the medical retreat program in accordance with the present invention;

FIG. 3 is a flow diagram of a nutrition plan for a member patient;

FIG. 4 is a flow diagram of a body activity plan for a member patient; and

FIG. 5 is a alternative medical treatment plan for a member patient.

### DETAILED DESCRIPTION THE PREFERRED EMBODIMENT

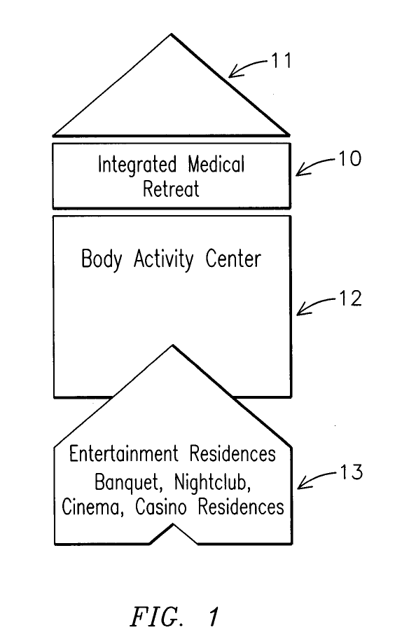

Referring to FIG. 1, the present invention is directed towards an integrated medical retreat 10 placed adjacent to a village resort 11 which includes lodging for people using the medical retreat 10 as well as entertainment, restaurants, boutiques and shops. The medical retreat also has a sports facility and activity center 12 which would include a gymnasium along with a heated pool, facilities for tennis, yoga, pilates, aerobics, Tai Chi, jogging, walking and the like and might generally be provided with golf facilities, boating and fishing. In addition there would be an entertainment and residences area 13 placed adjacent to the medical retreat 10 and sports facility 12 which includes banquet facilities, a nightclub, cinema, and can include a casino, and residences. The juxtaposition of these facilities is specifically designed to enhance the enjoyment of members or patients utilizing the medical retreat in the rejuvenating and restructuring of a patient's lifestyle while the patient is going through full medical exams to restructure their lifestyles in accordance with a plan specifically tailored to that particular individual member.

The operation of the integrated medical retreat and resort is set up in the nature of a club which provides equity membership in the integrated medical rejuvenation center which features a diagnostic and scientific clinic along with alternative medical choices and a gymnasium and fitness center and a spa and beauty treatment facility. A member acquires a membership in the club which provides the member with equity ownership entitling the member to vote on matters affecting the resort. A member buys this membership in the club which then provides him with the use of the health resort by the member and his family for up to six weeks within a one year period. A membership can be sold or inherited. In addition to the use of the medical retreat, it allows a member to have lodging in the adjacent village resort at a discounted members rate.

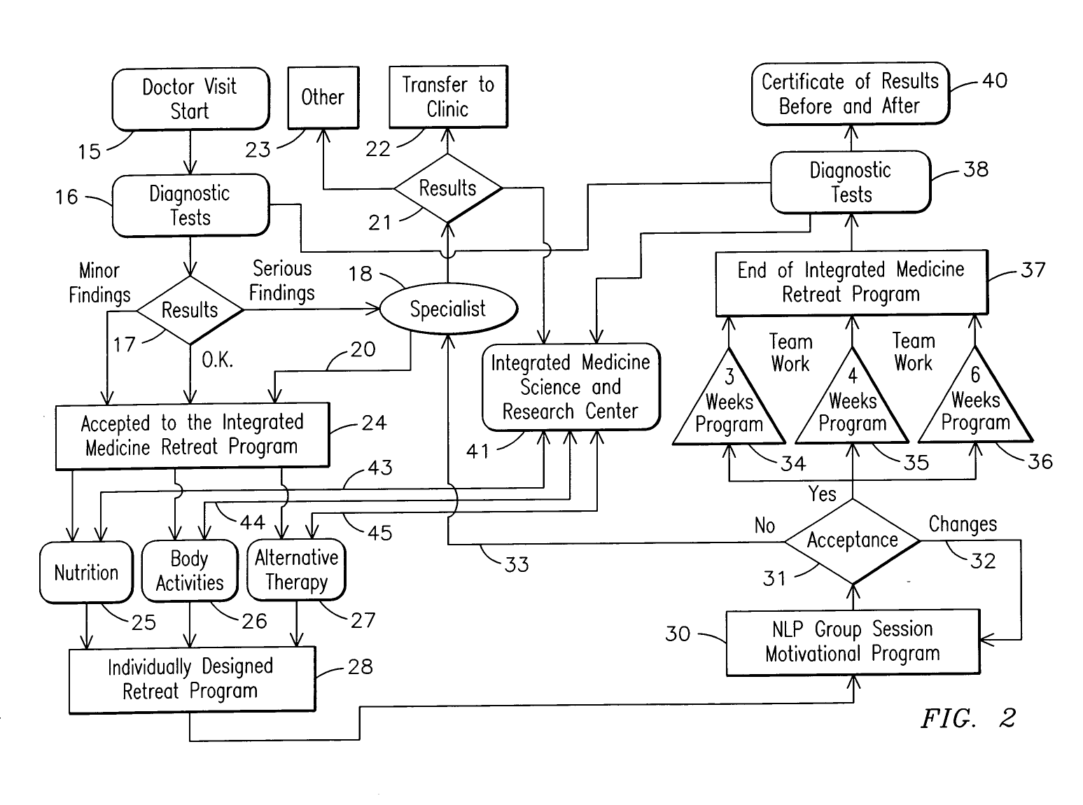

The integrated medical retreat and resort concept is illustrated in the flow diagram of FIG. 2. The medical retreat is in one central medical facility or building and starts with a doctor's visit 15 where the member/patient can provide a complete medical history and obtain a physical exam and is given a full series of diagnostic tests 16 which includes complete blood work to first establish that the member is healthy enough and free of serious disease before continuing with the life/health program. The diagnostic center provides for a complete physical exam including an EKG, CAT, MRA testing as well as cardio and pulmonary evaluation, metabolic analysis, and a complete blood evaluation and other tests as recommended by the physician. The results 17 will determine that the member patient is either healthy enough to proceed with the integrated medical retreat program, or if it finds any serious medical problems, the member is then turned over to a specialist dealing with his particular problem. The specialist 18 can then treat the patient and send him on through a transfer 20 or back to the beginning of the integrated retreat program or, depending on the results 21, the member patient can be transferred to a medical clinic 22 or other disposition 23. Once a member/patient is accepted 24 into the integrated medicine retreat program, the results of the diagnostic tests and physical exam as well as his medical history and his normal nutritional preferences and his exercise or physical activity preferences are incorporated into a computer for determining a program for the specific individual.

The programs provided include a nutritional plan 25, a body activities plan 26 and an alternate therapy plan 27. Each of these plans provide a computer and expert designed retreat and lifestyle program 28. The member patient then, utilizing these plans, attends a motivational program 30 provided by the retreat center which he can accept 31 or determine changes 32. If he does not accept the program, he then goes back to a specialist for dealing with a specialized medical problem. The program has been formatted for a three week 34, four week 35 or six week 36 program to rejuvenate the individual to improve his medical fitness for a longer and healthier life at the end of the retreat program 37. After the three to six week program, the member patient goes through a series of diagnostic tests 38 which are compared with the diagnostic tests 16 upon entering the retreat program to determine the improvements that have been made by the program. He then receives a certificate of the before and after results 40. The integrated medical retreat includes a science and research center 41 which receives the results through the line 42 of diagnostic tests 16 and the exit diagnostic test 38 and also receives an input from the derived nutrition program 25 and the body activities program 26 and the alternative therapy program 27. The three to six week retreat program includes a tailored plan for use in the activity center 12 as well as the tailored nutrition program and the motivational program.

While going through the retreat program, the member individual is able to utilize the village resort 11 lodging facilities and entertainment and restaurants and is able to utilize the entertainment and residences section 13 including the cinema, nightclub, banquet and casino, as desired.

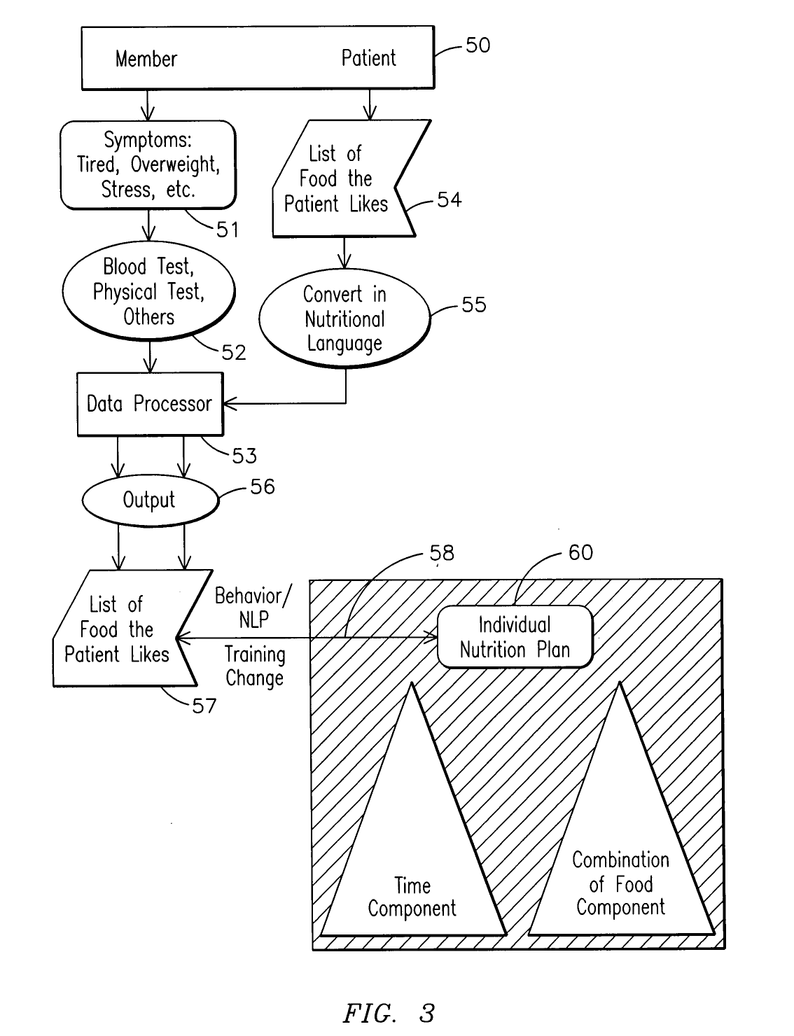

The derivation of a nutritional plan, is more clearly illustrative in FIG. 3 in which the member patient 50 has his symptoms and physical characteristics 51, such as being tired, overweight, stressed or the like, and the results of all of his blood tests and physical tests 52 fed into a data processor 53. In addition, the patient has a list of foods 54 which includes foods that the patient has indicated in his preferences which is converted into nutritional language 55 and fed into the computer 53. The output 56 from the data processor 53 produces a list of foods and diet 57 that the patient likes but has been nutritionally adjusted to improve his general health. The patient then undergoes behavior trained change 58 for his specific individual nutrition plan 60.

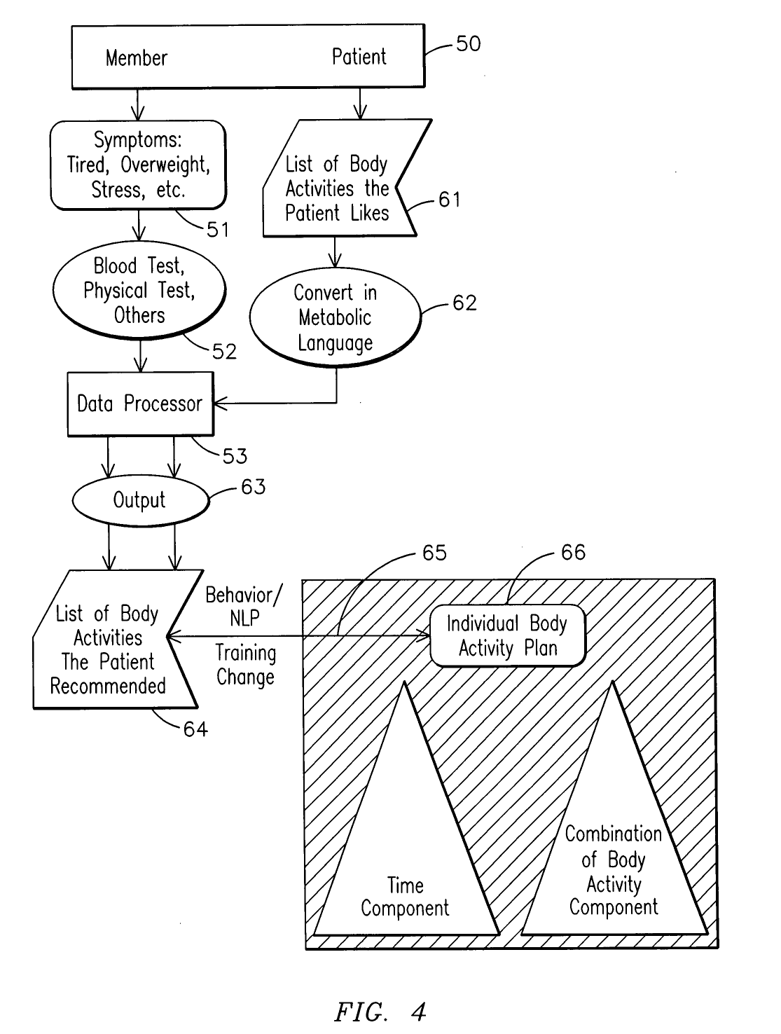

In FIG. 4, the body activity plan 26 uses the patient 50 symptoms 51 along with his blood tests 52 and a developed list of his preferred personal activities 61, which list is converted into a metabolic language 62, which is fed into the data processor 53 to produce an output 63 having a list of body activities 64. These recommendations to the patient provide sufficient activities to improve the health of the individual. The patient then goes through behavior training 65 to tailor his individual body activities 66 for the improved health and lifestyle changes that are being adopted for the three to six week retreat program.

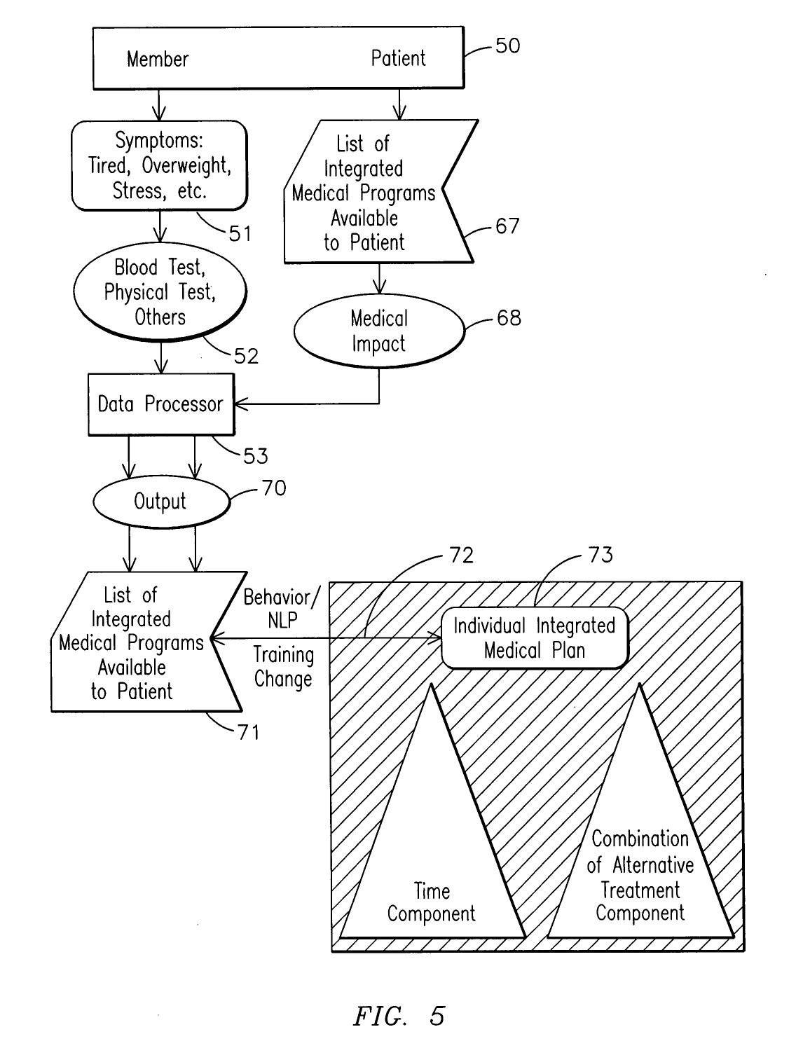

Turning to FIG. 5, an alternative therapy plan 27 for the member patient 50 is entered, along with a list of his symptoms 51 and the results from his blood tests and physical exam 52 into the data processor 53. Also, the patient enters a list 67 of the integrated medical programs available to the patient from which the medical impact 68 can be determined and fed into the data processor 53. The output 70 from the data processor 53 produces a list 71 of an integrated medical program for the patient which allows him to go through a behavior training change 72 and the production of an integrated medical plan 73.

The integrated medical retreat, as set forth, not only provides the services outlines but while a member patient or his family member is going through a retreat program, they simultaneously have made available to them preferential treatment within the facility and reduced fees for plastic surgery procedures and other procedures to enhance the appearance or beauty of the individual. This might include tummy tucks, breast enlargement, breast lifts, chemical peels, collagen/fat injections, derma-abrasion, ear surgery, laser eye surgery, face lifts, facial implants, forehead lift, hair replacement surgery, laser facial resurfacing, liposuction, male breast reduction, nose surgery, aesthetic dental surgery and implants. All surgical procedures are performed by medical specialists. The membership program also provides for other beauty services provided in a European style spa including sports massages, body wraps, scrubs and the like. Thus, an individual can take a medical retreat from three to six weeks during which time lifestyle changes are instituted to improve the patient's health for a healthy longer life while simultaneously obtaining plastic surgery and beauty treatments to improve their overall and improving their overall stamina and health. In the process, a member is able to have diets related to their normal diets, exercise and activities in accordance with their preferences while enjoying first class lifestyle in the lodging and entertainment of the adjacent entertainment facilities.

I claim:

1. An integrated medical clinic and retreat owner-member treatment process comprising the steps of:
acquiring equity ownership in a medical clinic and membership in a clinic associated medical retreat program to become an owner-member;
processing entry of owner-member into the medical clinic;
doctor's examination of owner-member;
diagnostic testing of owner-member;
designing an owner-member wellness program for entering the medical retreat program using diagnostic test results and Doctor's examination including nutrition, activities and therapy plans for owner-member during tenure in medical retreat;
entering the medical retreat program by owner-member for a predetermined period of time to follow said designed wellness program for owner-member; and
exit diagnostic testing for evaluating owner-member's change during said medical retreat, whereby a person can improve his health through an owner-membership program in a medical clinic and associated medical retreat program.

2. The integrated medical clinic and retreat process for owner-member in accordance with claim 1 including the step of referring the owner-member to a specialist responsive to his diagnostic testing results prior to entering the medical retreat program.

3. The integrated medical clinic and retreat process for owner-member in accordance with claim 1 in which the step of entering the retreat program for a predetermined period includes entering the retreat program for three to six weeks.

4. The integrated medical clinic and retreat process for owner-member in accordance with claim 1 in which the step of designing an owner-member wellness program includes designing physical activities in accordance with owner-member's interests to provide the necessary physical activity for an owner-member's lifestyle.

5. The integrated medical clinic and retreat process for owner-member in accordance with claim 4 in which the step of designing an owner-member wellness program includes designing a nutrition regimen in accordance with owner-member's tastes.

6. The integrated medical clinic and retreat process for owner-member in accordance with claim 5 in which the step of designing an owner-member wellness program including a motivational program.

7. The integrated medical clinic and retreat process for owner-member in accordance with claim 6 in which the step of designing an owner-member wellness program includes designing an alternative therapy program.
